(19) United States
(12) Patent Application Publication
(10) Pub. No.: US 2020/0315594 A1
(43) Pub. Date: Oct. 8, 2020

(54) MULTIPLE LUMEN DEVICE

(71) Applicant: ADN International, LLC, New York, NY (US)

(72) Inventors: Andrew Blitzer, New York, NY (US); Marshall Strome, New York, NY (US)

(73) Assignee: ADN International, LLC, New York, NY (US)

(21) Appl. No.: 16/300,920

(22) PCT Filed: May 12, 2017
(86) PCT No.: PCT/US2017/032463

§ 371 Date: Nov. 12, 2018

Related U.S. Application Data
(63) Continuation of application No. 15/363,080, filed on Nov. 29, 2016, now abandoned.
(60) Provisional application No. 62/367,670, filed on Jul. 28, 2016.
(60) Provisional application No. 62/335,209, filed on May 12, 2016.

(51) Int. Cl. A61B 10/02; A61B 5/06; A61B 1/00; A61M 25/10; A61B 1/015; A61B 5/00; A61M 25/00

(52) U.S. Cl. CPC A61B 10/02; A61B 5/065; A61B 1/00082; A61M 25/1002; A61B 1/015; A61B 1/00135; A61M 2025/1088; A61B 5/0084; A61B 5/6853; A61M 25/00; A61M 2025/105; A61M 2025/1004; A61B 1/00103

(57) ABSTRACT

A triple lumen expandable device designed for, in some embodiments, obtaining tissue from the aerodigestive tract is provided. The device may have internal and external folds and a tissue collection surface for collecting a tissue sample from a body lumen, such as the nose or throat. The device includes a camera and lumens for enhanced functionality. The present invention is also directed to methods of collecting a tissue sample using the devices, described herein.

### RELATED APPLICATIONS

This application claims priority under 35 U.S.C. § 119(e) to U.S. Provisional Application No. 62/367,670, filed Jul. 28, 2016 entitled “SINGLE EXTRUSION TRIPLE LUMEN DEVICE,” and U.S. Provisional Application No. 62/335,209 filed May 12, 2016, entitled “IMPROVED EXPANDABLE DEVICE FOR TISSUE COLLECTION FROM AN AERODIGESTIVE BODY LUMEN”, and under 35 U.S.C. § 120 to U.S. patent application Ser. No. 15/363,080 filed Nov. 29, 2016, entitled “SINGLE EXTRUSION TRIPLE LUMEN DEVICE” the entire contents of which are hereby incorporated by reference.

### FIELD OF THE INVENTION

The present invention relates to a multiple lumen device designed for collecting a tissue sample from a body lumen, such as the nose or throat and having improved tissue collection capabilities, as well as for treating and visualizing tissues within a body lumen. The device may include a camera and lumens for enhanced functionality and related methods.

### BACKGROUND OF INVENTION

Devices used for collecting tissue samples from the aerodigestive tract of a patient have not provided adequate clinical sampling of the entire circumference of the lumen. This lack of circumferential sampling often leads to sampling errors. Additionally, such existing devices often cause the tissue sample from being contaminated by neighboring tissue collected en route to and from the site of the suspected diseased tissue.

### SUMMARY OF INVENTION

In some aspects the invention is a triple lumen expandable device. In some embodiments it is a single extrusion device. The device has a catheter body having an outer surface sized to fit within a body lumen and extending distally from a proximal end and optionally having a handle positioned at the proximal end, one or more regions of the outer surface having a tissue collection surface, an expandable device positioned over at least a portion of the outer surface, wherein when the expandable device is deflated the outer surface has internal folds and external folds, wherein the tissue collection surface is present on one or more internal folds of the outer surface and is not present on one or more external folds, wherein the catheter body has at least three lumens, each having a distal opening at a distal end of the catheter, the first lumen having a distal opening within the expandable device, the second and third lumens having a distal opening distal to the expandable device. In some embodiments the device further comprises a camera positioned on the catheter distal to the expandable device. The camera in some embodiments is positioned on the end of the third lumen.

The first lumen, in some embodiments is a hollow compartment for transferring a gas or liquid to the expandable device to inflate the expandable device. In other embodiments the second lumen is a flushing channel. In yet other embodiments the third lumen is a camera channel. In other embodiments, the third lumen has a transparent end.

In some embodiments the distal opening of the second lumen is positioned proximal to the distal opening of the third lumen. In other embodiments the camera has an anti-mucous coating.

In some embodiments the device is a cytology device for the collection and retention of tissue samples from within an individual.

In certain embodiments, the expandable device is a balloon. In some embodiments, the balloon is made of latex, silicone elastomer, butadiene/acrylonitride copolymers, copolyesters, ethylene vinylacetate (EVB) polymers, ethylene/acrylic copolymers, ethylene/propylene copolymers, polyalkylacrylate polymers, polybutadiene, polybutylene, polyethylene, polyisobutylene, polyisoprene, polyurethane, styrenebutadiene copolymers, and styrene-ethylene/butylene-styrene, polyesters, polyolefins, polyamides, polyvinyl chloride, or a combination thereof. In certain embodiments, the balloon has one or more inflatable compartments. In some embodiments, the balloon has a shape selected from the group consisting of round, conical, oblong, and tissue specific. In some embodiments, the tissue specific shape is a shape that is an approximate mirror image of a body lumen.

In some embodiments, the tissue collection surface of the expandable device is an abrasive surface. The abrasive surface may be made of any abrasive, non-toxic material. In particular embodiments, the abrasive surface is a coating of particulate. In certain embodiments, the particulate is made of silica, a biocompatible plastic, a biopolymer (e.g., polycaprolactone (PCA), polyhydroxyalkanoate (PHA), polyhydroxybutanoate (PHB), or polyhydroxybutyrate-valerate (PHBV)), or a combination thereof.

In other embodiments, the tissue collection surface is on alternating internal folds. In particular embodiments, the tissue collection surface forms a pattern on the outer surface of the inflated expandable device. In some embodiments the tissue collection surface is not present on any external folds, and in other embodiments the tissue collection surface is present on all internal folds.

In certain embodiments, the expandable device described herein has an expanded configuration and a contracted configuration, wherein the internal and external folds are pleated when the device is in the contracted configuration and unpleated when the device is in the expanded configuration. In certain embodiments, the folds are corrugated in the contracted configuration.

In some embodiments, the device is attached to a tube or channel. In certain embodiments, an instrument is advanced through the tube or channel prior to contracting the expandable device. The instrument may be a laser fiber, a cytology brush, an applicator, a needle, forceps, or a blade. The tube or channel may be, for instance, an endoscope or part of an endoscope.

In some embodiments, an agent is delivered to the individual prior to contracting the expandable device. The agent can be delivered to the individual through the tube or channel, or the agent can be coated on the surface of the expandable device. In some embodiments, the agent is coated on the internal folds of the expandable device. In some embodiments, the agent can be a therapeutic agent, a diagnostic agent or an imaging agent.

In other aspects the invention is a method of collecting tissue from an individual. The method involves (a) advancing a triple lumen expandable device as described herein, wherein the expandable device is deflated within a body lumen of an individual to a tissue collection site; (b) expanding the expandable device at the tissue collection site to unfurl at least some of the folds so that the tissue collection surface contacts tissue of the body lumen; (c) collecting tissue on the tissue collection surface of the expandable device; (d) deflating the expandable device; and (e) removing the deflated device from the individual.

In some embodiments the body lumen is selected from the group consisting of pharynx, larynx, oropharynx, nasopharynx, nasal cavity, nose, throat, trachea, esophagus, urethra, bladder, colon, cervix, and duodenum. In other embodiments the step of collecting tissue involves rotating the expandable device.

Certain aspects described herein relate to a balloon cytology device, comprising: a balloon having an outer surface sized to fit within an esophagus, one or more regions of the outer surface having a tissue collection surface; and a support member having a proximal end region and a distal end region, wherein the support member is connected to the balloon at the distal end region and wherein the minimal length of the support member between the proximal end region and the distal end region is 10 cm, wherein the support member includes a hollow compartment for transferring a gas or liquid to the balloon to inflate the balloon.

In certain embodiments, the apparatus of any of the foregoing embodiments comprises an actuator at the proximal end region. In some embodiments, the actuator is a syringe.

In certain embodiments, the apparatus does not include a cover which covers part or all of the expandable device.

In other embodiments, the expandable device is a balloon comprised of a thin material. In yet other embodiments the catheter body further comprises a stiffening agent, such as a wire in the lumen or embedded in the wall of the catheter body. In yet other embodiments the catheter body has a distal tip extending out beyond the expandable device. In other embodiments the distal tip comprises a soft material.

In some aspects the invention is a disposable single or multiple extrusion multiple lumen device, a flexible catheter body having an outer surface sized to fit within a body lumen and extending distally from a proximal end to a distal end, wherein the catheter body has at least two lumens, each having a distal opening at or near the distal end of the catheter, wherein the first lumen is a hollow compartment for transferring components from the proximal to distal ends of the catheter, the second lumen being a flushing channel, a visualization device, optionally a camera, positioned on the catheter distal to the expandable device, and a light source in proximity to the visualization device.

In some embodiments the disposable multiple lumen device further includes a handle fixed to the proximal end of the flexible catheter body. The handle may include a housing that encloses a steering mechanism comprising a rotating toothed gear. In some embodiments the handle housing contains a control element, wherein a portion of the control element is exposed to a proximal end of the handle such that the control element can be manipulated single-handedly. In other embodiments the flexible catheter body is detachably and directly connected at its proximal end to a distal end of the handle and includes at least one guide wire disposed therein, wherein the at least one guide wire extends from the proximal end of the flexible catheter body and is constructed to fit into a distal end portion of the steering mechanism housed in the handle such that the at least one guide wire can be manipulated by the steering mechanism to move the flexible catheter body in one of at least four different directions.

In some embodiments the handle does not include any gears. In other embodiments the handle is disposable. The handle is not detachably fixed to the flexible catheter body in other embodiments.

A third lumen that is a hollow compartment for transferring a gas or liquid to an expandable device attached to the catheter body, to inflate the expandable device is provided in other embodiments of the invention. In some embodiments the expandable device is a balloon.

In yet other embodiments the catheter body has an outer surface sized to fit within an esophagus, pharynx, larynx, trachea, urethra, bladder, colon, cervix, or duodenum.

In some embodiments the catheter body is made of silicone elastomer.

In other embodiments the first lumen is constructed and arranged to deliver a therapeutic agent, diagnostic agent, or imaging agent to a tissue selected from the group consisting of an esophagus, pharynx, larynx, trachea, urethra, bladder, colon, cervix, or duodenum.

The visualization device in some embodiments is a camera which is positioned near the end of the second lumen. Optionally, the camera has an anti-mucous coating.

In some embodiments an insufflation valve positioned on the handle housing and operably connected to the third lumen. In yet other embodiments an irrigation/suction valve positioned on the handle housing and operably connected to the second lumen.

A method of treating an individual is provided in other aspects of the invention. The method involves (a) advancing a disposable multiple lumen device of the invention into a tissue selected from an esophagus, pharynx, larynx, trachea, urethra, bladder, colon, cervix, or duodenum of the individual; (b) visualizing a delivery site in the tissue using the visualization device and light source; (c) treating the delivery site by delivering through the first lumen of the catheter body an agent or a surgical instrument to the delivery site in order to treat the individual; and (d) removing the disposable multiple lumen device from the individual.

In some embodiments the agent is a therapeutic agent, a diagnostic agent or an imaging agent. In yet other embodiments the step of treating the delivery site involves the delivery of both an agent and a surgical instrument.

Still other aspects provided herein are directed to a kit, comprising: (a) the apparatus of any one of the foregoing embodiments; and (b) instructions or direction for obtaining instructions for using the apparatus. In certain embodiments, components (a) and (b) are arranged in a container.

In some aspects the invention is a device, having a catheter body having an outer surface sized to fit within a body lumen and extending distally from a proximal end, an expandable device positioned over at least a portion of the outer surface, wherein when the expandable device is inflated it has a substantially cylindrical shape with tapered ends, and wherein when the expandable device is deflated the outer surface has internal folds and external folds, wherein a textured surface is present on one or more internal folds of the outer surface and is not present on one or more external folds, and wherein the textured surface is comprised of 1-500 strips of a tissue collection material on each internal fold.

In some embodiments the textured surface is comprised of 2-50 strips of a textured material on each internal fold. In other embodiments the tissue collection surface is comprised of 3-9 strips of a textured material on each internal fold. In yet other embodiments each internal fold includes 2 side walls and a bottom wall and wherein 3 strips of textured material are present on at least one the two side walls and/or the bottom wall of each internal fold. In some embodiments each of the 3 strips are arranged linearly with a space between strips. In other embodiments two of the 3 strips are 8-15 mm in length and the third strip is 15-29 mm in length. In some embodiments two of the 3 strips are 10 mm in length and the third strip is 25 mm in length. In some embodiments the strip of 25 mm in length is positioned between the two 10 mm strips.

The strips may be made of a clear or white material. In some embodiments the strips are polyolefin low profile hook strips.

In some embodiments the textured surface is a tissue collection surface. In some embodiments the textured surface is a drug delivery surface. In some embodiments the textured surface is an abrasive surface.

In some embodiments the expandable device is made of a clear material. In other embodiments the expandable device is made of a medical grade silicone. In other embodiments the strips are attached to the expandable device using a light cure Loctite 5055.

The expandable device in some embodiments has 10-15 internal folds and in other embodiments has 12 internal folds. Each internal fold may have 2 walls bonded to 1 or more strips. In some embodiments the expandable device has 24-84 strips bonded on internal folds. In other embodiments the expandable device has 36 strips bonded on internal folds.

When the expandable device is inflated all of the strips may be exposed to the surface and arranged for contact with tissue. In some embodiments at least 80%, 85%, 90%, or 95% of the surface of the inflated expandable device is cylindrical.

In some embodiments when the tapered ends are elliptical in end shaped.

In other embodiments 30%-90%, 50%-80%, or 30%-40% of the internal folds have a textured surface.

The internal folds in some embodiments are 40-60 mm in diameter, 0.5-3 mm in height and 0.05-0.2 mm depth. In other embodiments the internal folds are 50 mm in diameter, 1 mm in height and 0.1 mm depth.

In some embodiments the internal folds have blunt end walls which are perpendicular to side and bottom walls of the internal fold.

The device may also include a visualization device positioned on the catheter distal to the expandable device. In some embodiments the visualization device is a camera. The camera may be a 1.4 mm camera. The visualization device may also include a video processor unit to process a signal received from the camera and to provide video output to a receiver. In some embodiments the video processor unit further comprises a fiber coupled light source.

In some embodiments the catheter body has at least three lumens, each having a distal opening at a distal end of the catheter, the first lumen having a distal opening within the expandable device, the second and third lumens having a distal opening distal to the expandable device. In other embodiments a handle is positioned at the proximal end of the catheter.

In yet other embodiments the drug delivery surface is coated with an agent. The agent may be a therapeutic agent, diagnostic agent, or imaging agent.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

FIG. 1 is a schematic of an embodiment of the tissue collection device having an expandable device: deflated, side view.

FIGS. 2A-2B schematize an embodiment of the device in cross section, deflated within a body lumen (FIG. 2A), and inflated within a body lumen (FIG. 2B).

FIGS. 3A-3B schematize embodiments of the distal end of the device showing the second lumen and camera (FIG. 3A) and the second and third lumens (FIG. 3B).

FIG. 4A-4B schematize one embodiment of the distal dual lumen extrusion region of the device in side view (FIG. 4A) and cross section (FIG. 4B).

FIG. 5 schematizes one embodiment of a cytology collection apparatus, including an inflated device, first, second and third lumens and a camera.

FIG. 6 is a schematic of an embodiment of the device with a handle, side view.

FIGS. 7A-7B schematize cross-sectional views of an embodiment of the device, at the distal end of the handle housing (FIG. 7A) and a detailed view of the catheter tube portion marked as A in FIG. 7A (FIG. 7B).

FIG. 8 is a schematic of an embodiment of the device showing the handle connected to the catheter body and including valves and channels.

FIG. 9 is a schematic of an embodiment of the device at the proximal end of the handle housing in cross section view.

FIG. 10 schematizes one embodiment of the device in side view and cross section of the side view.

FIG. 11A-11C is a set of schematics of an embodiment of the expandable device including side view showing Sections A-A and B-B (FIG. 11A), a side view of Section A-A (FIG. 11B) and a cross-sectional view of Section B-B (FIG. 11C).

FIG. 12A-12B is a schematic (FIG. 12A) of an embodiment of the expandable device showing wall indentations or inner and outer folds and a photograph (FIG. 12B) of an embodiment of the expandable device showing wall indentations or inner and outer folds without any textured surface or strips.

FIG. 13A-13D is a set of photographs of embodiments of the device including side views of the expandable device having rows of textured surfaces or strips with 7 strips per inner fold (FIG. 13A), 3 even size strips per inner fold (FIG. 13B), or 3 differentially sized strips per inner fold (FIG. 13C) and a full view of the device including the expandable device, the catheter and handle (FIG. 13D).

FIG. 14A-14E is a set of photographs of an expandable device having non-optimal strips including a protruding strip (FIG. 14A), wrinkled strips (FIGS. 14B and 14C), strips causing a diamond shape (FIG. 14D), and strips causing a protruding side of an expandable device (FIG. 14E).

FIG. 15 is a photograph of an expandable device having optimally situated strips and a substantially cylindrical structure with tapered sides.

FIG. 16 is a photograph of an embodiment of an expandable device having a camera linked to an external video processor, a light source and a fluid source or syringe.

FIG. 17A-17D is a set of photographs of embodiments of a device including the catheter (FIG. 17A), the expandable device and camera (FIG. 17B), an insertion port (FIG. 17C), and a proximal end with syringe and luer port (FIG. 17D).

FIG. 18A-18D is a set of photographs of a products visualized using an exemplary camera for use on the device. The photographs include a bar code (FIG. 18A), 500 micron beads (FIG. 18B), oatmeal grain (FIG. 18C), and 200 micron beads (FIG. 18D).

### DETAILED DESCRIPTION OF INVENTION

Provided herein are methods and devices for collecting a tissue sample from a body such as a cylindrical lumen in the upper aerodigestive tract. The aerodigestive tract includes for instance, nasopharynx, nose, throat, airway, or esophagus. Obtaining tissue from the aerodigestive tract remains a technical challenge. A number of devices have been designed to achieve tissue sampling in this area of the body. However, the existing devices have many limitations. The devices of the invention provide a safe disposable solution for accurate and directed non-invasive sampling of tissue from this area of the body. Tissue sampling from cylindrical lumens is typically accompanied by some degree of sampling error, as it is difficult to collect cells from the entire circumference of, for example, an esophagus. By providing a device having the structural properties described herein and which expands to the walls of the lumen, it is possible to obtain a true sampling of the lumen, using a disposable device.

The devices are expandable (e.g., inflatable) and generally have folded regions, at least some of which include a tissue collection surface for capturing the tissue once the device is expanded. The device is inserted into the aerodigestive tract. The device may also incorporate a flexible visualization device such as a camera to enable direct visualization. The camera is activated and used to direct the device to the appropriate position for tissue sampling. The camera may be a disposable camera or other visualization device attached to the tissue collection device. Alternatively the camera may be a reusable camera which is removably attached to or associated with the tissue collection device. The device also includes several lumens including a lumen which allows for air or liquid to be passed through the device in order to clear the camera for better viewing.

The visualization device may be a camera or other imaging device, or other radiant energy sensor for radiant temperature, thermal imaging, reflected energy spectral analyzer, color, texture or fluorescence analysis. Recently developed commercial subminiature cameras for vision may be used for instance. The imaging can also be achieved via an imaging fiber optics bundle as a sensor at the tip and locating a camera at the other end of the fiber bundle remote from the patient.

The camera when attached to the device can be interconnected with a low power radio frequency transmitter so as to transmit images recorded by the camera to a display module to the user of the device i.e., in the operating room or physician's office. Transmitters of this type are commercially available and can be adapted for the intended use. The image captured by the camera can be digitized and recorded. The image recorded by the camera can also be displayed real time on a video monitor through the wireless interconnection. The ease of a wireless transmission system in the confines of a medical procedure avoids the likelihood of a patient and attending health care providers from becoming entangled with cords and wires. In one embodiment, the camera can be located at the distal end of the tissue collection device. A light source may be provided so as to provide better images transmitted from the camera. In one embodiment the light source comprises at least one diode strategically placed on the device in order to provide ample but focused light on the area to be imaged by the camera.

The camera may furthermore have a reflective exterior to improve illumination light guiding. The reflective surface may have mirror finish or diffuse characteristics to achieve desired light patterns. The second lumen 40 between the camera 44 and the tip of the camera tube 46 may optionally also function as a fluid channel passing fluid or gas. This fluid can serve as a means of keeping the camera lens area purged and clean.

A computer can be coupled to the light source and the camera in order to control, for example, camera, light emission, and image display functions. More specifically, each computer used with the system can include a processor configured to activate the light, collect imaging data from the camera, analyze the imaging data, display the imaging data or outputs indicative of the analysis, and/or perform other operations (e.g., by executing a software program stored on the computer).

In some embodiments the visualization device comprises a camera tube or third lumen 46 with a distal end and a proximal end. In some embodiments, the camera tube or third lumen 46 is sealed at the distal end with a transparent material. The diameter of camera tube 46 is designed in such a way that a camera can be inserted inside of the camera tube through an opening at the proximal end and moved down the camera tube toward the distal end, so that the camera transmits continuously images obtained through the transparent material. The length of the camera tube can vary and it can be adjusted dependent on the length of the device. As the camera does not come in contact with a patient, there is no need to sterilize the camera and the same camera can be reused in many applications. Thus, the same camera can be switched between different devices and patients. The continuous visualization of the patient is in real time and enables remote monitoring as well.

The visualization device 198 can be further equipped with a portable light 204 which can be either built-in the camera or it can be built-in the camera tube or remain as an external source and is connected to a light source 202. Alternatively, a light source can remain outside the camera tube on the proximal end, but still be placed such that the light source sheds light inside of the camera tube. The camera 44 has a tip 42 and may have its own light source. As the visualization device transmits images from a patient in real time, it can be used for guiding the device for proper placement.

At least in some applications, the camera is a digital camera equipped with a chip and it collects and transmits images continuously. The camera can be connected wirelessly or hard-wired with a computer network which collects and analyzes images obtained by the camera. This arrangement permits for remote, continuous and real time monitoring of the device during placement and after-placement in a patient. Thus, an accurate and rapid placement of the device can be achieved. Further and because the visualization device continues to acquire images after the device is placed inside of a patient i.e. during cell collection, the patient can be monitored in real time for adverse reactions such as bleeding, airway obstruction, shifting or malfunctioning, etc. of the device and other reactions.

In some embodiments the camera has an anti-mucous coating. The anti-mucous coating is useful for enhancing the visualization while the camera is in the body.

At the desired site of tissue collection, the device is expanded, such that the folds unfurl and are exposed to the tissue walls. The tissue collection surface on the now unfolded regions of the device is then allowed to contact the tissue. The tissue collection surface is configured in a way such that it is capable of dislodging the tissue from the tissue wall and capturing it on the surface. The device is then reduced or deflated such that the regions of the device having tissue collection surfaces are folded and face internal, such that they are no longer exposed to the tissue walls. The device may then be removed from the individual. As the device is being removed from the individual, the tissue collected using the device is protected from the body environment. Once the device is outside of the body the tissue can be removed from the tissue collection surface using any known methods in the art, for instance by using a buffered (e.g., PBS (phosphate buffered saline)) wash.

The expandable device described herein has an outer surface sized to fit within a cylindrical body lumen of an individual. A cylindrical body lumen, as used herein, refers to any space or cavity in the upper aerodigestive tract formed by a tubular or tubular-like organ. The term cylindrical is not used in this context to indicate a perfect cylindrical shape, but rather to indicate the tubular organs of the aerodigestive tract. A tubular or tubular-like organ is one that has an external surface forming a space or lumen positioned within the body but that is exposed to the outside of the body. For example, organs of the aerodigestive tract having a cylindrical body lumen include but are not limited to a nose or throat, including the pharynx, larynx, oropharynx, nasopharynx, nasal cavity, trachea, and esophagus of an individual. In a particular embodiment, the device is sized to fit within an esophagus of an individual.

An individual, as discussed herein, refers to a human. Preferably the human is a patient in need of cytological analysis.

The device of the invention is useful for collecting tissue from an individual. Tissue, as used herein, refers to a sample of material from a subject, including at least one cell. Preferably the tissue sample is composed primarily of cells that are obtained from the lumen of the individual. For instance, this may include epithelial cells or any cells present in a tumor or abnormal growth occurring in any of the foregoing body lumens.

The tissue may be removed from the expandable device using any methods known to those of skill in the art. The tissue samples can then be processed in a number of ways. For example the tissue may have been collected for the purpose of detecting the presence or absence of cancer cells in the tissue. A number of tumors of the head and neck are associated with the squamous epithelium of the mucosal lining in the nose and throat. Additionally, the cells lining the esophagus can develop into esophageal cancer. These and other cells can be sampled easily and effectively using the device of the invention to provide, for example, a cytology tool in routine examination, or a diagnostic tool in cancers or other abnormal growths of the upper aerodigestive tract.

In some instances, the tissue being sampled has a mucosal surface. It may be desirable to remove the mucous layer prior to (or at the same time) as tissue sampling. It is possible to achieve this by pretreating with or applying a mucolytic agent on the expandable device. Mucolytic agents include but are not limited to acetylcysteine, ambroxol, bromhexine, carbocisteine, domiodol, dornase alfa, eprazinone, erdosteine, letosteine, mesna, neltenexine, sobrerol, stepronin, and tiopronin.

The outer surface of the expandable device has a tissue collection surface. A tissue collection surface, as used herein refers to a region of the expandable device that is configured to capture tissue. It may be configured to capture tissue by having a charged or otherwise sticky surface. Alternatively or additionally, it is configured to capture tissue by having a rough surface that dislodges the tissue from the lumen. The configuration of the surface may be dictated by the types of materials used to produce or coat the surface as well as the shape of the material making up or coating the surface. Materials used in the preparation of the expandable device as well as the tissue collection surface are described in more detail below.

The tissue collection surface is positioned on the folds of the device such that when the device is expanded at the desired site in the body and the tissue collection surface contacts the walls of the lumen, the tissue collection surface will dislodge and capture tissue at the site at one or more contact points along the lumen. The tissue collection surface may contact only a portion of the lumen or alternatively may contact the entire circumference of the lumen. The contact between the tissue collection surface and the lumen results in a transfer of at least some tissue from the lumen to the tissue collection surface. In order to enhance the transfer of the tissue, the expandable device may be moved relative to the lumen. The movement may involve any range of motion that assists the transfer of the tissue to the tissue collection surface. For instance, the expandable device may be moved horizontally and/or vertically, or it may be rotated, as discussed in more detail below.

The expandable device also has some internal and external folds. The folds may be present when the device is deflated, and when the device is inflated the amount and extent of the folds are decreased. If the expandable device is completely inflated the appearance of the folds may be absent. It is desirable in some cases to have at least some of the tissue collection surface located on the internal folds. The tissue collection surface covers, in some embodiments, at least 50%, 60%, 70%, 80%, 90% or 95% of the internal folds. In other instances, the tissue collection surface covers 100% of the internal surfaces. In a deflated or partially deflated configuration the tissue collection surface on the internal folds would be protected from exposure to the lumen.

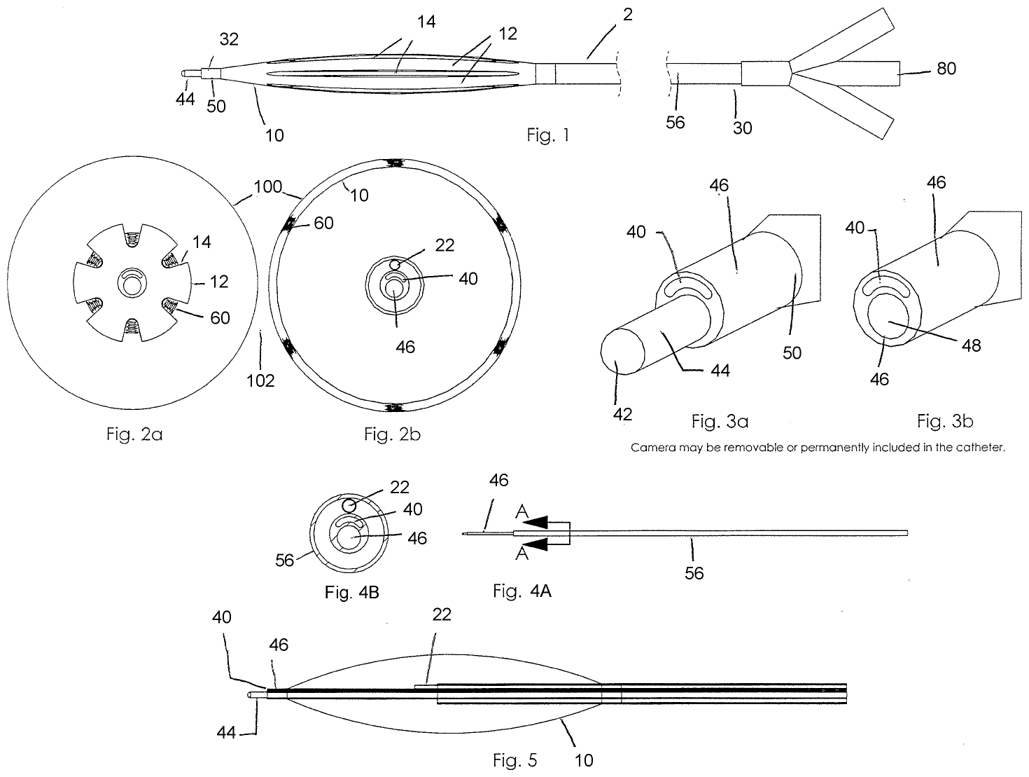

The folds may have any type of configuration or pattern. Types of folds include but are not limited to half fold, tri-fold, gate fold, Z fold, parallel fold, accordion fold, quarter fold, pleats, reverse folds, squash folds half/tri fold, and tri/half hold. FIG. 1 provides examples of an expandable device of the invention having pleat folds in a deflated configuration. In FIG. 1 the expandable device 10 is shown from a side view, such that the external folds 12 and internal folds 14 can be observed.

The number of internal folds on the expandable device may range from about two to about thirty. In some embodiments, the expandable device has at least two internal folds, while in other embodiments, it has at least ten internal folds. In yet other embodiments, the expandable device has at least fifteen or at least twenty internal folds, or more. In particular embodiments, the expandable device has more than 30 folds. The number of folds (internal and external) depends on the size and shape of the device, which depends on the size and shape of the body lumen in which the device is used. The shape of the device may be any shape as long as it is capable of collecting cells in an inflated state. The shape of the device may be, for instance, round, conical, or oblong. Alternatively, the shape of the device is tissue specific. For example, the device may approximately mirror the image of the cylindrical lumen (e.g., esophagus) from which the tissue sample is collected. The particular shape and dimensions of the expandable device may be selected as required for its specific purpose and for the particular tissue collection site at which it will be used. For example, expandable devices configured for introduction into the throat (e.g. esophagus or trachea) may have diameters of up to about 25 mm, or more. In some embodiments, the diameter of the device expands to about 10 to about 30 mm in diameter, while in other embodiments, the diameter of the device expands to about 15 to about 20 mm in diameter. The lengths of the expandable devices described herein vary widely, depending on the application. For example, the length of the expandable device can be up to about 100 mm, or more. In some embodiments, the expandable device is about 10 to about 100 mm, whereas in other embodiments, it is about 20 to about 50 mm in length.

Expandable, as used in the context of expandable device, refers to a material which is capable of being transitioned from a compact form (e.g., deflated) to an expanded form (e.g., inflated). Expandable devices useful according to the invention include but are not limited to balloons, balloon expandable collectors or self-expandable collectors. A balloon is an inflatable, flexible bag made of elastic or elastic-like (e.g., polymer-based elastic) that expands as it is filled with a gas, such as helium, hydrogen, nitrous oxide, oxygen, or air. When a balloon is expanded, typically the material from which the balloon is made (e.g., rubber, latex, polychloroprene, a nylon fabric) expands (stretches as a result of its elasticity).

A balloon expandable collector is a composite balloon and cover collection device such that when the balloon portion is inflated, the cover of the device contacts a lumen for tissue collection. An example of a balloon expandable collector is a non-balloon material covering a balloon. The non-balloon material may be physically attached, partially or fully, to the balloon or may unattached to the balloon.

A self-expandable collector is a self-expandable device that does not require inflation by a gas. For instance, the self-expandable collector may be a sac or pouch made of material that is not elastic or elastic-like (e.g., polymer-based elastic or memory metal such as nitinol) and is not necessarily stretched when the device is expanded. Alternatively it may be made of an elastic material that is constrained to a small size but which expands when deconstrained. In other embodiments, it may be a composite of materials, one of which having memory (e.g., nitinol or plastic spring). In certain embodiments, a self-expandable collector requires delivery by catheter or other cylindrical support structure. In such embodiments, the self-expandable device is positioned within the catheter as it is advanced to the tissue collection site, then deployed from the catheter to self-expand for tissue collection.

In certain embodiments, the expandable device may be made of latex, silicone elastomer, butadiene/acrylonitride copolymers, copolyesters, ethylene vinylacetate (EVB) polymers, ethylene/acrylic copolymers, ethylene/propylene copolymers, polyalkylacrylate polymers, polybutadiene, polybutylene, polyethylene, polyisobutylene, polyisoprene, polyurethane, styrenebutadiene copolymers, and styrene-ethylene/butylene-styrene, polyesters, polyolefins, polyamides, polyvinyl chloride, or equivalent or combination thereof. Other materials are well known to the skilled artisan.

The tissue collection surface may be formed from the same material or from a different material than the other non-tissue collection surfaces of the expandable devices. In some embodiments, the tissue collection surface is an abrasive material (particulate). In particular embodiments, the abrasive surface is a coating of particulate. In certain embodiments, the particulate is made of silica, a biocompatible plastic, a biopolymer, or combinations thereof. Examples of biopolymers include polycaprolactone (PCA), polyhydroxyalkanoate (PHA), polyhydroxybutanoate (PHB), and polyhydroxybutyrate-valerate (PHBV). In some embodiments, the particulate is made of granules. In certain embodiments, the granular size of the particulate is about 5 to about 500 microns. In other embodiments, the granular size of the particulate is about 25, or about 50 microns. The thickness of the tissue collection surface may be the same as, thinner than, or thicker than the expandable device material. In some embodiments, the tissue collection surface is thicker than the device material. Even in such embodiments where the thickness of the tissue collection surface exceeds that of the device material, the tissue collection surface is protected from exposure to the environment by the folds of the device such that the tissue collection surface is not contaminated during protraction and retraction of the device.

The tissue collection surface may form a pattern, for example, a horizontal or vertical pattern. A pattern may be an organized or random arrangement of the abrasive particulate.

Depending on the device and the purpose of the device, the tissue collection surface is present on at least one of the internal folds. For instance, the tissue collection surface may be on a single internal fold, all internal folds, alternating internal folds or any combination thereof. The tissue collection surface may be present on at least 50%, 60%, 70%, 80%, 90% or 95% of the internal folds. In some embodiments, the tissue collection surface is on 100% of the internal folds.

The tissue collection surface may also be present on some of the external folds. For instance the tissue collection surface is present on less than 40%, 30%, 20%, 10%, 5%, or 1% of the external folds. In some embodiments the tissue collection surface is absent altogether from the surface of the external folds. An example of a configuration having the tissue collection surfaces present on all the internal folds and not on any of the external folds is shown in FIG. 2B. In such embodiments, when the device is inflated within a body lumen, the tissue collection surfaces 60 of the internal folds 14 are exposed and contact the wall of the lumen (FIG. 2B), but when the device is deflated the tissue collection surface is retracted and protected from subsequent contact with the walls of the lumen (FIG. 2A). In this way, the collected tissue sample does not become contaminated with neighboring cells (and conversely the neighboring cells are not damaged) when the expandable device is removed from the body lumen.

The tissue collection surface may or may not be on any external folds. If it is desirable to collect cells from as many surfaces as possible the collection surface may be present on one or more external folds. By having tissue collection surfaces present on the external folds as well as the internal folds, the different sections of the device may be used to collect tissue from different areas of the body. For instance, the external folds will be exposed to the tissue as the device passes through different lumen. It is also exposed to the tissue at the specified collection area, where the device is expanded. When the device is removed from the patient the external folds having a tissue collection surface will have a mixture of cells from different tissues and the internal folds will only have cells from the tissue sampled while the device was expanded.

Alternatively, the tissue collection surface may also be present on an external surface under conditions when it is not desirable to collect cells outside of the target area. In this case, the device can be used with a protective cover. The expandable device may be placed inside the protective covering and advanced into a body lumen to a tissue collection site in this configuration. At the tissue collection site, the expandable device is deployed from the protective covering, and subsequently expanded, fully or partially, to contact the wall of the lumen. The tissue sample is collected, the device is contracted, retracted back into the protective covering, and then removed from the body lumen.

A protective cover, as used herein, refers to a structural element sized to enclose part or all of the expandable device, such that the expandable device is shielded from contact with surfaces. The protective cover may be made of a flexible material such that it may be folded back on itself or otherwise collapsed to expose the expandable device. Alternatively, it may be made from an inflexible material. In such a case it could simply be slid off the expandable device, or the expandable device could be moved out from the protective cover at the tissue collection area of the lumen.

In some embodiments, the protective cover is a tube, such as a catheter, plastic stylet, or other covering used to deliver a medical device to a body lumen. A protective cover may optionally be used with any configuration of expandable device described herein.

Alternatively, the expandable device may be attached to a tube or channel, through which an object such as equipment or an agent (e.g., therapeutic agent such as medicine) may be delivered. For example, an endoscope may be advanced through the tube to visualize the area around the area around the expandable device. Alternatively the tube or channel may be an endoscope or part of an endoscope. An endoscope is an instrument used to examine the interior of a hollow organ or cavity of the body. Typically, endoscopes are inserted directly into the organ. In some embodiments, an endoscope is comprised of a rigid or flexible tube, a light delivery system to illuminate the organ or object under inspection (the light source can be outside of the body and the light can be directed via an optical fiber system), a lens system that transmits an image (still or motion) to a viewer from an objective lens to the viewer (e.g., a relay lens system in the case of rigid endoscopes or a bundle of fiber optics in the case of a fiberscope), and an eyepiece. In some embodiments, the endoscope is also attached to an additional tube or channel to allow entry of equipment such as medical instruments or manipulators.

In some embodiments, equipment is delivered through a tube or channel of the expandable device. Such equipment includes, but is not limited to, a laser fiber, a cytology brush, an applicator, a needle, forceps, and a blade.

Any one of the foregoing devices and embodiments may be used in a method of collecting cells from an individual, as described herein. Such methods include (a) advancing a deflated (contracted) expandable device having internal and external folds and a tissue collection surface on one or more of the internal folds to a collection site within a body lumen of an individual; (b) expanding (inflating) the expandable device at the collection site to unfurl at least some of the folds so that the tissue collection surface contacts tissue of the body lumen; (c) collecting tissue on the tissue collection surface of the expandable device; (d) contracting (deflating) the expandable device; and (e) removing the contracted expandable device from the individual.

In certain embodiments, collecting tissue involves rotating the fully expanded device. Rotating refers to making a circular movement around an imaginary center (rotation) axis. Rotating the device permits sloughing and subsequent collection of the tissue by the tissue collection surface. The device is considered to be fully expanded when the tissue collection surface contacts the wall of the lumen at the collection site. In other embodiments, collecting tissue involves moving the fully inflated device up and down (along the longitudinal axis of the lumen), such that motion permits sloughing and subsequent collection of the tissue by the tissue collection surface.

In some embodiments, the expandable device is delivered to the tissue collection site in the individual without a stent or protective cover. In other embodiments, however, a protective cover is used. For example, if the tissue collection device is on the external folds, then the device is delivered within a protective cover so that the tissue collection surface does not contact neighboring tissue en route to the tissue collection site.

It is also possible to use the device of the invention for the delivery of agents. Agents include, for instance, therapeutic agents, diagnostic agents, and imaging agents (e.g., labeled compounds). Therefore, the invention contemplates a device having an agent linked to the device. The agent may be coated on the surface of the expandable device, on the interior and/or exterior folds. Alternatively, the agent may be enclosed in a pouch or other enclosure that can be activated to open and release the agent.

The agents may be naturally occurring or non-naturally occurring. Naturally occurring agents include those capable of being synthesized by the subjects on whom the expandable device is used. Non-naturally occurring are those that do not exist in nature normally, whether produced by plant, animal, microbe or other living organism.

The agent may be without limitation a chemical compound including a small molecule, a protein, a polypeptide, a peptide, a nucleic acid, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form. The invention further contemplates the loading of more than one type of agent on the expandable device.

One class of agents is peptide-based agents such as (single or multi-chain) proteins and peptides. Examples include antibodies, single chain antibodies, antibody fragments, enzymes, co-factors, receptors, ligands, transcription factors and other regulatory factors, some antigens (as discussed below), cytokines, chemokines, hormones, and the like.

Another class of agents that can be delivered using the expandable device of the invention includes chemical compounds.

A variety of agents that are currently used for therapeutic or diagnostic purposes can be delivered according to the invention and these include without limitation imaging agents, immunomodulatory agents such as immunostimulatory agents and immunoinhibitory agents (e.g., cyclosporine), antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, analgesics, opioids, enzyme inhibitors, neurotoxins, hypnotics, anti-histamines, lubricants, tranquilizers, anti-convulsants, muscle relaxants, anti-Parkinson agents, anti-spasmodics, muscle contractants including channel blockers, miotics and anti-cholinergics, anti-glaucoma compounds, modulators of cell-extracellular matrix interactions including cell growth inhibitors and anti-adhesion molecules, vasodilating agents, inhibitors of DNA, RNA or protein synthesis, anti-hypertensives, anti-pyretics, steroidal and non-steroidal anti-inflammatory agents, anti-angiogenic factors, anti-secretory factors, anticoagulants and/or antithrombotic agents, local anesthetics, prostaglandins, targeting agents, neurotransmitters, proteins, cell response modifiers, and vaccines.

Imaging Agents.

As used herein, an imaging agent is an agent that emits signal directly or indirectly thereby allowing its detection in vivo. Imaging agents such as contrast agents and radioactive agents that can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include ²⁰¹T1, gamma-emitting radionuclide 99 mTc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203 Pb, and 11In; imaging agents for in vivo fluorescence imaging. In other embodiments, the agent to be delivered is conjugated, or fused to, or mixed or combined with an imaging agent.

Immunostimulatory Agents.

As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod, imidazoquinoline, resiquimod, nucleic acids comprising an unmethylated CpG dinucleotide, monophosphoryl lipid A or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

Antigens.

The antigen may be without limitation a cancer antigen, a self-antigen, a microbial antigen, an allergen, or an environmental antigen. The antigen may be peptide, lipid, or carbohydrate in nature, but it is not so limited.

Cancer Antigens.

A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, amp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Microbial Antigens.

Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

Allergens.

An allergen is an agent that can induce an allergic or asthmatic response in a subject. Allergens include without limitation pollens, insect venoms, animal dander dust, fungal spores and drugs (e.g. penicillin). Examples of natural, animal and plant allergens include but are not limited to proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elation); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Adjuvants.

The adjuvant may be without limitation saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic) Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod, resiquimod). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages. In these latter instances, the adjuvant may be incorporated or be an integral part of the nucleic acid gel and will be released as the gel is degraded.

Immunoinhibitory Agents.

As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants.

Growth Factors.

The expandable device may be coated with growth factors including without limitation VEGF-A, VEGF-C P1GF, KDR, EGF, HGF, FGF, angiopoietin-1, cytokines, endothelial nitric oxide synthases eNOS and iNOS, G-CSF, GM-CSF, VEGF, aFGF, SCF (c-kit ligand), bFGF, TNF, heme oxygenase, AKT (serine-threonine kinase), HIFa hypoxia inducible factor), Del-1 (developmental embryonic locus-1), NOS (nitric oxide synthase), BMP's (bone morphogenic proteins), SERCA2a (sarcoplasmic reticulum calcium ATPase), beta-2-adrenergic receptor, SDF-1, MCP-1, other chemokines, interleukins and combinations thereof.

Anti-Cancer Agents.

As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids. or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin (DOXIL); Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Flurocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-Ia; Interferon gamma-Ib; Iproplatin; Irinotecan; Lanreotide; Lenalidomide (REVLIMID, REVIMID); Letrozole; Leuprolide; Liarozole; Lometrexol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemetrexed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimetrexate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

The anti-cancer agent may be an enzyme inhibitor including without limitation tyrosine kinase inhibitor, a CDK inhibitor, a MAP kinase inhibitor, or an EGFR inhibitor. The tyrosine kinase inhibitor may be without limitation Genistein (4′,5,7 trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy5-(2,5-Dihydroxybenzylamino)-2-hydroxybenzoic acid. The CDK inhibitor may be without limitation p21, p2′7, p5′7, p15, p16, p18, or p19. The MAP kinase inhibitor may be without limitation KY12420 (C₂₃H₂₄O₈), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WHI-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a VEGF inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-C5, IOR-T6 (anti-CD1), IOR EGF/R3, celogovab (ONCOSCINT OV103), epratuzumab (LYMPHOCIDE), pemtumomab (THERAGYN), and Gliomab-H (indicated for brain cancer, melanoma).

Anti-Infective Agents.

The agent may be an anti-infective agent including without limitation an anti-bacterial agent, an anti-viral agent, an anti-parasitic agent, an anti-fungal agent, and an anti-mycobacterial agent.

Anti-bacterial agents may be without limitation β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin.

Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-viral agents may be without limitation amantidine and rimantadine, ribivarin, acyclovir, vidarabine, trifluorothymidine, ganciclovir, zidovudine, retinovir, and interferons.

Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Anti-fungal agents may be without limitation imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhbitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin).

Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride:Bithionolate Sodium; Bromchlorenone; Carbamide Peroxide; Cetalkonium Chloride; Cetylpyridinium Chloride:Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene:Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Other Agents.

The agent may be without limitation adrenergic agent; adrenocortical steroid; adrenocortical suppressant; alcohol deterrent; aldosterone antagonist; ammonia detoxicant; amino acid; amylotropic lateral sclerosis agent; anabolic; analeptic; analgesic; androgen; anesthetic; anorectic; anorexic; anterior pituitary activator; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic including (3-adrenergic agonists, methylxanthines, mast cell stabilizing agents, anticholinergics, adrenocortical steroids such as glucocorticoids; anti-atherosclerotic; anticholelithic; anticholelithogenic; anticholinergic; anticoagulant; anticoccidal; anticonvulsant; antidepressant; antidiabetic; antidiarrheal; antidiuretic; antidote; antidyskinetic; anti-emetic; anti-epileptic; anti-estrogen; antifibrinolytic; antiglaucoma; antihemorrhagic; antihemorrheologic; antihistamine; antihyperlipidemic; antihyperlipoproteinemic; antihypertensive; antihypotensive; anti-infective; anti-inflammatory; antikeratinizing agent; antimigraine; antimitotic; antimycotic; antinauseant; antineutropenic; antiobsessional agent; antioxidant; antiparkinsonian; antiperistaltic; antipneumocystic; antiprostatic hypertrophy agent; antiprotozoal; antipruritic; antipsoriatic; antipsychotic; antirheumatic; antischistosomal; antiseborrheic; antisecretory; antispasmodic; antithrombotic; antitus sive; anti-ulcerative; anti-urolithic; appetite suppressant; blood glucose regulator; bone resorption inhibitor; bronchodilator; carbonic anhydrase inhibitor; cardiac depressant; cardioprotectant; cardiotonic; cardiovascular agent; cerebral ischemia agent; choleretic; cholinergic; cholinergic agonist; cholinesterase deactivator; coccidiostat; cognition adjuvant; cognition enhancer; conjunctivitis agent; contrast agent; depressant; diagnostic aid; diuretic; dopaminergic agent; ectoparasiticide; emetic; enzyme inhibitor; estrogen; estrogen receptor agonist; fibrinolytic; fluorescent agent; free oxygen radical scavenger; gastric acid suppressant; gastrointestinal motility effector; geriatric agent; glucocorticoid; gonad-stimulating principle; hair growth stimulant; hemostatic; herbal active agent; histamine H2 receptor antagonists; hormone; hypocholesterolemic; hypoglycemic; hypolipidemic; hypotensive; HMGCoA reductase inhibitor; impotence therapy adjunct; inflammatory bowel disease agent; keratolytic; LHRH agonist; liver disorder agent; luteolysin; memory adjuvant; mental performance enhancer; mineral; mood regulator; mucolytic; mucosal protective agent; multiple sclerosis agent; mydriatic; nasal decongestant; neuroleptic; neuromuscular blocking agent; neuroprotective; NMDA antagonist; non-hormonal sterol derivative; nutrient; oxytocic; Paget's disease agent; plasminogen activator; platelet activating factor antagonist; platelet aggregation inhibitor; post-stroke and post-head trauma agents; progestin; prostaglandin; prostate growth inhibitor; prothyrotropin; psychotropic; radioactive agent; relaxant; rhinitis agent; scabicide; sclerosing agent; sedative; sedative-hypnotic; selective adenosine A1 antagonist; sequestering agents; serotonin antagonist; serotonin inhibitor; serotonin receptor antagonist; steroid; stimulant; suppressant; thyroid hormone; thyroid inhibitor; thyromimetic; tranquilizer; unstable angina agent; uricosuric; vasoconstrictor; vasodilator; vulnerary; wound healing agent; or xanthine oxidase inhibitor.

In certain embodiments, provided herein is an apparatus comprising the expandable device 10 of any of the foregoing embodiments and a support member or tube 56. A support member or tube 56 as used herein, is a flexible element that is connected on one end to the expandable device, and optionally on the other end to a guide element or handle. The support member is used to guide the expandable device to and from the body lumen during the sampling process. The support member must be flexible in order to navigate the inside of the individual's body such that it is able to deliver the expandable device to the sampling site.

The expandable device may be fixedly attached (not removable) to the support member, while in other embodiments, it is removable. The support member may be made of any material having a tensile strength sufficient to support movement of the expandable device through body lumens. Examples of material useful in the construction of a support member include but are not limited to a hydrogel, silicone, polyethylene, polypropylene, polyurethane, polycaprolactone, polytetrafluoroethylene (PTFE), copolymers, or a combination thereof. The support member may be a catheter or stylet. In some embodiments, the support member may be or may comprise a guide-wire. In some embodiments, the support member has a proximal end region and a distal end region and the expandable device (e.g., balloon) is located at the distal end region. In other embodiments, the support member comprises a guide at the distal end region.

In certain embodiments, the support member exceeds the length of the expandable device. For instance, the support member may extend beyond both the proximal and distal ends of the device. In some embodiments, the expandable device is attached (fixedly or removably) to the proximal end of the support member such that the distal end 60 of the support member extends beyond the expandable device. Alternatively, the expandable device may be attached (fixedly or removably) to the distal end of the support member such that that proximal end 62 of the support member or tube 56 extends beyond the expandable device 10. The distance between the proximal end region and the distal end region of the support member (length) can vary greatly, as long as it is within a range useable in a human body. For instance, the length may be about 2 cm to 40 cm, 5 to 20 cm, or 5 cm to 15 cm. In particular embodiments, the distance is about 10 cm.

The device has an expanded (inflated) configuration and a contracted (deflated) configuration. The device may also assume an intermediate configuration. When deployed at a tissue collection site, the device can be partially expanded or fully expanded. In its fully expanded state, the internal and external folds are no longer pleated. Other than with self-expanding devices, the apparatus will involve a mechanism for expanding the device. It typically is expanded using a gas or liquid.

In order to achieve this, the support member or tube may include a hollow compartment or lumen for transferring a gas or liquid to the device to expand the device. Examples of gases used herein include oxygen, nitrogen, carbon dioxide, and water vapor. Examples of liquids include water-based, alcohol-based, or gel-like liquids. In certain embodiments, the gas or liquid is transferred through the hollow compartment to the center of the device, thereby expanding the device as the volume of the gas or liquid increases to fill the device.

In some embodiments, the apparatus comprises an actuator at the proximal end region. An actuator refers to a device for moving or controlling movement of the expandable device or entire apparatus. The actuator may be mechanical or electrical. For example, an actuator may be used to advance the expandable device to the tissue collection site. They actuator may also be used to inflate and deflate the expandable device. In some embodiments where a protective covering is used, the actuator is used to deploy the expandable device from the protective cover. In certain embodiments, the actuator is a syringe. A syringe may be used to deliver a liquid or gas to the expandable device, thereby expanding the device, either fully or partially. The syringe may be connected with the expandable device via the support member, a catheter, or other hose. Alternatively, the movement of the expandable device may be by hand and does not employ an actuator.

FIG. 1 illustrates an embodiment of the expandable device. The expandable device 10 is shown in its deflated configuration. The expandable device may have multiple folds, for example, 6 folds, 8 folds, 12 folds, 24 folds, or more. In its deflated (contracted) configuration, the internal folds 14 of the device are pulled in and away from the surface of the device, while the external folds 12 remain exposed at the surface. In its inflated (expanded) configuration, the internal folds 14 of the device are forced outward (unfurled) to allow for contact with the tissue of the lumen wall.

FIG. 2 illustrates several embodiments of the device of the invention in a cross sectional view. The device is shown in its deflated configuration (FIG. 2A) and its inflated configuration (FIG. 2B). The device has a tissue collection surface 60 on the surface of each internal fold 14. In its deflated configuration, the internal folds 14 are tucked between the external folds 12, and the tissue collection surface 60 is protected from exposure to the body lumen wall 100 and surrounding environment 102 (FIG. 2A). When the device 10 is expanded (e.g., fully expanded), the internal folds 14 are forced outward, placing the tissue collection surface 60 in direct contact with the internal wall (tissue) of the body lumen 100 (FIG. 2B). As the expandable device is moved (e.g., rotated or moved back and forth), the tissue is sloughed off of the lumen wall and becomes entrapped on or within the tissue collection surface 60. The expandable device is then deflated (contracted) into its corrugated, original configuration, such that the collected tissue is entrapped in the internal folds and protected from exposure to the external environment (FIG. 2A).

In certain embodiments, the device of the invention has three lumens. The first lumen is also referred to as the balloon inflation lumen 22. The first lumen has an internal opening through which, for example, air or gas can be delivered in order to inflate the expandable device. A hose (e.g., catheter) or other instrument may also be inserted through the lumen. In some embodiments, the opening is at the proximal end of the device 30, while in other embodiments, there is an opening at both the proximal and distal 32 ends of the device. The opening of the first lumen at the distal end of the device is within the expandable device. When air or gas is passed through the lumen the air or gas can accumulate in the expandable device and cause it to inflate. Air or gas may also be removed from the expandable device through the first lumen in order to deflate the expandable device.

A second lumen, also referred to as a flushing lumen 40, is shown in cross section in FIG. 2B. The flushing lumen has a distal end near to the end of the third lumen or camera tube 46. When fluid or gas is passed through the second lumen 40 the fluid or gas can clear away debris or fluid obstructing the camera view.

FIG. 1 also illustrates an embodiment of the expandable device 10 having a support member or handle attached to the device (either fixedly or removable attached). The support member or handle 52 may be a solid structure and may or may not have controls or gears for manipulating movement of the device or its components. It may be detachable.

In certain embodiments, delivery of a gas or liquid to the device 10 is through a lumen in the support member. The lumen may be perforated to allow exit of the gas or liquid, or the lumen may be open-ended within the device.

The apparatus of the invention may include an expandable device 10 attached to a support tube 56 at its proximal end (relative to the body lumen opening) and, optionally, to a handle 82. Alternatively it may be joined to a connector (e.g. valve) at its proximal end, and a syringe and plunger attached to the support tube 56 via as second connector 86. Prior to advancement, the plunger may be used to backfill the syringe with a gas or liquid, then the syringe is attached to the support tube 56 via the connector. Prior to advancement of the device 10 into a body lumen, the distal end of the support tube 56 is joined to the proximal end of the support member via a connector. Alternatively the hose is connected to the support member, either by slipping the proximal end opening of the support member over the distal end of the support tube 56 to form a seal, or by slipping the distal end opening of the support tube 56 over the proximal end opening of the support member.

The expandable device 10 and support tube 56 are then advanced into the lumen to the tissue collection site, guided by the distal end of the support member or by a guide wire (or by the device 10 itself). In order to determine the position of the tissue collection site the camera may be used. Once the device 10 is positioned at the tissue collection site, the plunger is slowly actuated, thereby releasing the gas or liquid such that it travels through the support tube 56 to the support member. In certain embodiments, the support member is perforated, allowing the gas or liquid to be released into the expandable device 10, thereby filling and inflating the device 10 such that the internal folds 14 unfurl permitting contact of the tissue collection surface with the wall of the lumen. The device 10 is then moved (e.g., rotated or moved back and forth), dislodging and collecting a tissue sample from the lumen wall. After the sample is collected, the device 10 is deflated by retracting the plunger, thereby drawing out the gas or air used to inflate the device. The device 10 and support tube 56 are then carefully removed from the lumen.

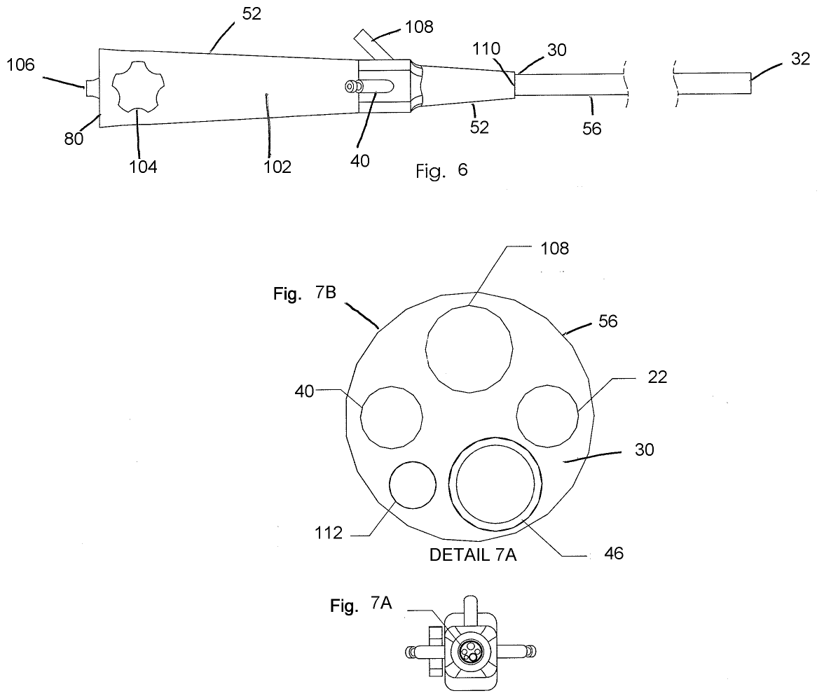

FIGS. 6-10 show several views of a disposable multiple lumen device of the invention. A disposable device is configured to be disposed of after a single use (e.g., not intended to be reused, restored, or re-sterilized). Disposable devices can be made from materials which are not designed to withstand multiple exposures to harsh treatments such as washing or sterilization. The disposable device may include a disposable catheter body connected to a reusable handle or alternatively both the handle and the catheter body may be disposable. When a reusable handle is used with a disposable catheter body, the handle and the disposable body can be configured such that they connect to one another directly or through a connector such as a connection chamber. An exemplary connection chamber 101 is shown in FIG. 10.

There have been a number of problems with sterility and sterilization of multiple use devices. For instance, long periods of sterility and brushing channels of scope devices does not guarantee sterility. Hospitals are also concerned with eliminating prions, which requires as much as 28 minutes in a sterilization unit. The longer wait times have two effects: increasing the wait time between procedures unless the institution buys more expensive scopes and decreasing the half-life of the instruments because of the increase heat exposure per procedure. The disposable scopes of the invention provide an advantage over the reusable devices facing these sterility issues.

A schematic of an embodiment of the device with a handle, side view is shown in FIG. 6. The handle 52 is connected to the catheter tube 56 at the proximal end 30 of the catheter tube 56. A deflection knob 104 is positioned near the distal end of the handle housing 102. At the distal end of the handle 80 a camera and light connection port 106 is positioned to provide a connection between the device and external equipment for the camera and light signals. A terminal portion of a flushing channel 40 and a first hollow lumen 108 are depicted extending from the handle 52.

In FIG. 7 a detailed cross-sectional view of an embodiment of the device, at the distal end of the handle housing 110 (FIG. 7A) and a detailed view of the catheter tube portion marked as A in FIG. 7A (FIG. 7B) is provided. FIG. 7A shows the cross section of the distal handle including the flushing lumen 40, the first hollow lumen 108, and the balloon inflation lumen 22. Each of the three lumens extends out of the handle. In the center of the handle a cross-sectional view is provided of the catheter body. This view is shown in more detail in FIG. 7B. A cross section of the flushing lumen 40, the first hollow lumen 108, and the balloon inflation lumen 22 as well as a tube or lumen 46 for the camera or camera wires and the light source 112 are shown.

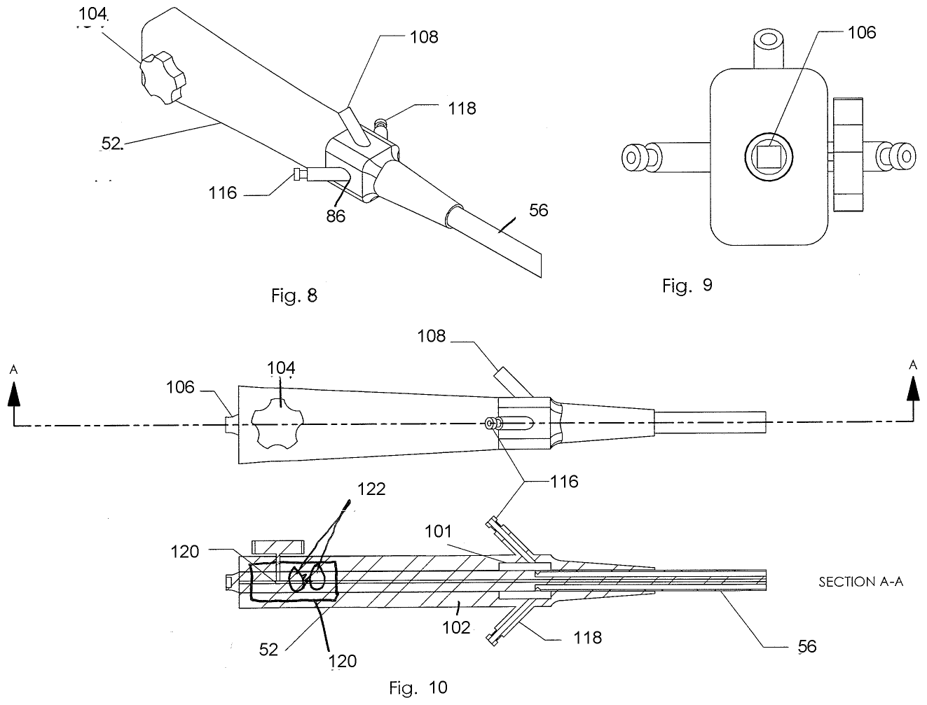

In FIG. 8 an alternate side view of the handle 52 connected to the catheter body and including valves and channels is shown. Irrigation/suction valve 116 and insufflation valve 118 are depicted near to the distal end of the handle 52. The proximal end of the handle housing 102 is shown in cross section in FIG. 9. The camera and light connection port 106 is shown centrally located.

A side view and cross section version of the side view of the entire device are shown in FIG. 10. As shown in the figure, the device includes a flexible catheter body 56 extending distally from a proximal end 30 to a distal end 32. The catheter body has several lumens, each having a distal opening at or near the distal end of the catheter and another opening exiting the handle 52. As shown in the figure the flushing lumen 40 includes an irrigation/suction valve 116 at the end. The balloon inflation lumen 22 includes an insufflation valve 118 at the end. A connection chamber 101 connects the handle to the proximal end of the catheter.

The disposable multiple lumen device further includes a handle fixed to the proximal end of the flexible catheter body. The handle 52 may include a housing 102 that encloses a steering mechanism 120 comprising a rotating toothed gear 122. In some embodiments the handle housing contains a control element such as a deflection knob 104, wherein a portion of the control element is exposed to a proximal end of the handle such that the control element can be manipulated single-handedly.

Thus, the handle assembly 112 may include a housing 102 that encloses a steering mechanism 118. The steering mechanism 118 may include a rotating toothed gear 122 carried on a shaft within the housing 102. The toothed gear 122 and control knob 104 may be attached to shaft by connectors such as splines. The toothed gear 122 may be seated for rotation between upper part and lower parts of housing. The control knob 104 may be seated against an O-ring or sealant mechanism, which seals the housing and also provides resistance against movement so that the catheter will remain in a selected position until a new position is selected by the physician.

In other embodiments the flexible catheter body is detachably and directly connected at its proximal end to a distal end of the handle and includes at least one guide wire 120 disposed therein, wherein the at least one guide wire 120 extends from the proximal end of the flexible catheter body and is constructed to fit into a distal end portion of the steering mechanism housed in the handle such that the at least one guide wire can be manipulated by the steering mechanism to move the flexible catheter body in one of at least four different directions. In other embodiments the handle may not include any gears.

The device described herein may be used, for instance, in a methods of treating an individual. The device may be used to enter a body lumen, visualize a desired tissue and deliver an agent such as a therapeutic agent to the tissue. Alternatively a surgical device may be manipulated at the tissue site using the device. Typically the tissue will be an esophagus, pharynx, larynx, trachea, urethra, bladder, colon, cervix, or duodenum of the individual.

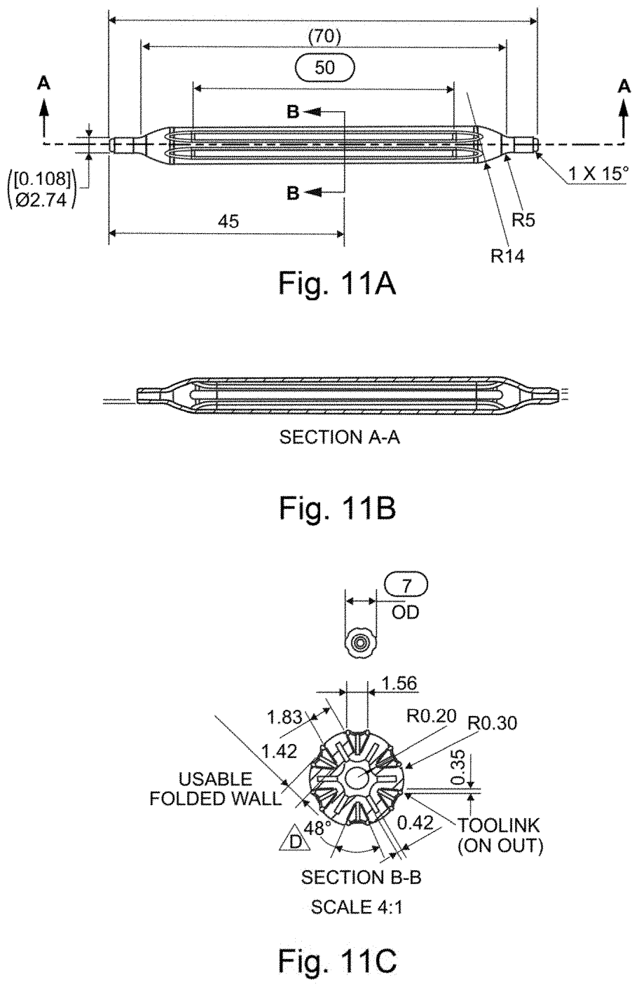

In some aspects the invention is a device, in some embodiments of which are shown in FIGS. 11-13 and 15-17. The device has a catheter body having an outer surface sized to fit within a body lumen and extending distally from a proximal end, an expandable device positioned over at least a portion of the outer surface, wherein when the expandable device is inflated it has a substantially cylindrical shape with tapered ends, and wherein when the expandable device is deflated the outer surface has internal folds and external folds, wherein a textured surface is present on one or more internal folds of the outer surface and is not present on one or more external folds, and wherein the textured surface is comprised of 1-500 strips of a tissue collection material on each internal fold.

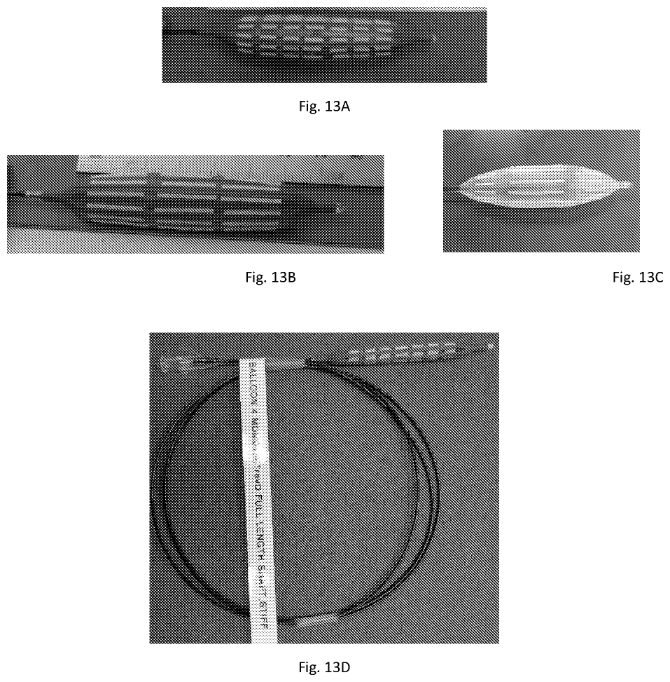

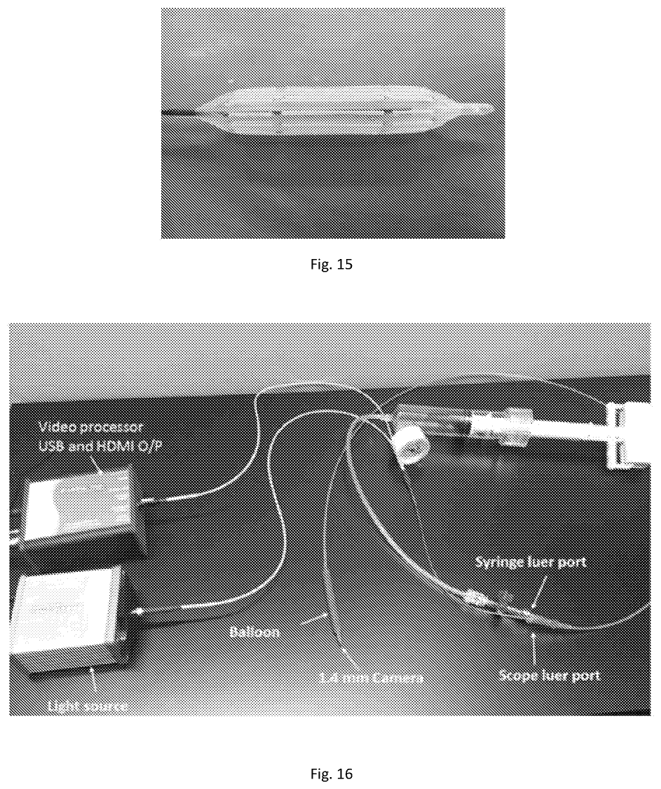

In some embodiments the textured surface is comprised of 1-200, 1-100, 2-100, 2-50, 3-100, 3-200, 3-36, 3-24, 3-12, 3-6, 4-200, 4-100, 4-50, 4-36, 5-200, 5-100, 5-50, 5-36, 6-100, 6-50, 6-25, 6-36, 7-100, 7-50, 7-36, 8-100, 8-50, 8-36, 9-100, 9-50, 9-36, 9-24, or 9-12 strips of a textured material on each internal fold. In other embodiments the tissue collection surface is comprised of 3-9 strips of a textured material on each internal fold. In yet other embodiments each internal fold includes 2 side walls and a bottom wall and wherein 3 strips of textured material are present on at least one the two side walls and/or the bottom wall of each internal fold. Preferably the strips are arranged linearly in the internal folds such that a space is left between the strips. The space between strips may be about 1-5, 1-10, 1-3, −1-4 or 1-2 mm in some embodiments. Examples of different orientations or arrangements of strips are shown in FIGS. 13 and 15. Each internal fold may have a single strip or multiple strips.

In one preferred embodiment shown in FIGS. 15 and 13C two of the 3 strips are 8-15 mm in length and the third strip is 15-29 mm in length. In some embodiments two of the 3 strips are 10 mm in length and the third strip is 25 mm in length. In some embodiments the strip of 25 mm in length is positioned between the two 10 mm strips. This embodiment is one example of a configuration that forms a substantially cylindrical shape, with tapered ends, when the expandable device is inflated.

A cylindrical shape as used herein refers to a geometrical figure with straight parallel sides and a circular or oval cross section. A substantially cylindrical shape as used herein refers to a geometrical figure with parallel sides in which at least 90% of the parallel sides are straight and a circular or oval cross section which has less than 10% variability in that structure. In some embodiments the substantially cylindrical shape is sausage shape. The purpose of the substantially cylindrical shape is to expose the maximum amount of the textured surface to body tissue when the expandable device is inflated. Thus, in some embodiments the expandable device has a substantially cylindrical shape when greater than 80% of the strips are exposed to the surface in a configuration that they would be able to interact with the body tissue in a body cavity or area. In some embodiments the expandable device has a substantially cylindrical shape when greater than 85%, 86%, 87%, 88%, 89%, 90%, 91%, 92%, 93%, 94%, 95%, 96%, 97%, 98%, 99%, or 100% of the strips are exposed to the surface in a configuration that they would be able to interact with the body tissue in a body cavity or area.

Various configurations are shown in FIG. 13A-13D. In FIG. 13A the expandable device has rows of textured surfaces or strips with 7 strips per inner fold. Other configurations include 3 even size strips per inner fold (FIG. 13B) and 3 differentially sized strips per inner fold (FIG. 13C). FIG. 15 is a photograph of an expandable device having optimally situated strips and a substantially cylindrical structure with tapered sides.

In some embodiments the textured surface is a tissue collection surface as described herein. In some embodiments the textured surface is a drug delivery surface. In yet other embodiments the drug delivery surface is coated with an agent. The agent may be a therapeutic agent, diagnostic agent, or imaging agent as described herein. In some embodiments the textured surface of the strips is an abrasive surface. The abrasive surface may be made of any material useful for collecting cells or delivering a drug or agent. The strips may be made of a clear or white material. In some embodiments the strips are polyolefin low profile hook strips.

In some embodiments the expandable device is made of a clear material. In other embodiments the expandable device is made of a medical grade silicone. Medical grade silicones are generally grouped into three categories: non implantable, short term implantable, and long-term implantable. Since the components of the invention are not being implanted any of these can be used. Materials approved by the FDA as Class V and VI can be considered medical grade. Most medical grade silicones are at least Class VI certified.

The strips or textured surfaces are attached to the expandable device using a material that is sufficiently flexible that it allows movement when the expandable device is inflated and strong enough to hold the strips on the medical grade silicone. Many glues do not have sufficient properties to achieve these goals. One material useful according to the invention is a light cure Loctite 5055.

The expandable device in some embodiments has 10-15 internal folds and in other embodiments has 12 internal folds. Each internal fold may have 2 walls bonded to 1 or more strips. In some embodiments the expandable device has 24-84 strips bonded on internal folds. In other embodiments the expandable device has 36 strips bonded on internal folds.

The internal folds in some embodiments are 40-60 mm in diameter, 0.5-3 mm in height and 0.05-0.2 mm depth. In other embodiments the internal folds are 50 mm in diameter, 1 mm in height and 0.1 mm depth. In other embodiments 30%-90%, 50%-80%, or 30%-40% of the internal folds have a textured surface. In some embodiments the internal folds have blunt end walls which are perpendicular to side and bottom walls of the internal fold.

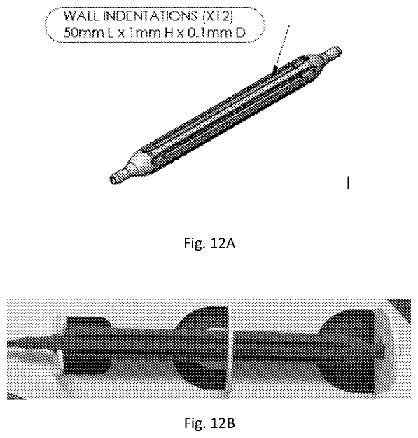

FIGS. 11A-11C and 12A-12B is a set of schematics of an embodiment of the expandable device including side view showing Sections A-A and B-B (FIG. 11A), a side view of Section A-A (FIG. 11B) and a cross-sectional view of Section B-B (FIG. 11C). T expandable device has wall indentations or inner 12 and outer 14 folds.

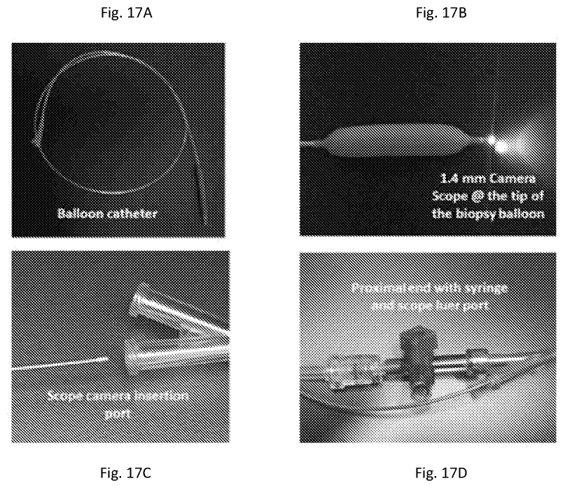

The device may also include a visualization device positioned on the catheter distal to the expandable device. Exemplary visualization device and related accessories are shown in FIGS. 16-18. In some embodiments the visualization device is a camera. The camera may be a 1.4 mm camera. The visualization device may also include a video processor unit to process a signal received from the camera and to provide video output to a receiver. In some embodiments the video processor unit further comprises a fiber coupled light source. FIG. 16 is a photograph of an embodiment of an expandable device having a camera linked to an external video processor, a light source and a fluid source or syringe. FIGS. 17A-17D is a set of photographs of embodiments of a device including the catheter (FIG. 17A), the expandable device and camera (FIG. 17B), an insertion port (FIG. 17C), and a proximal end with syringe and luer port (FIG. 17D).

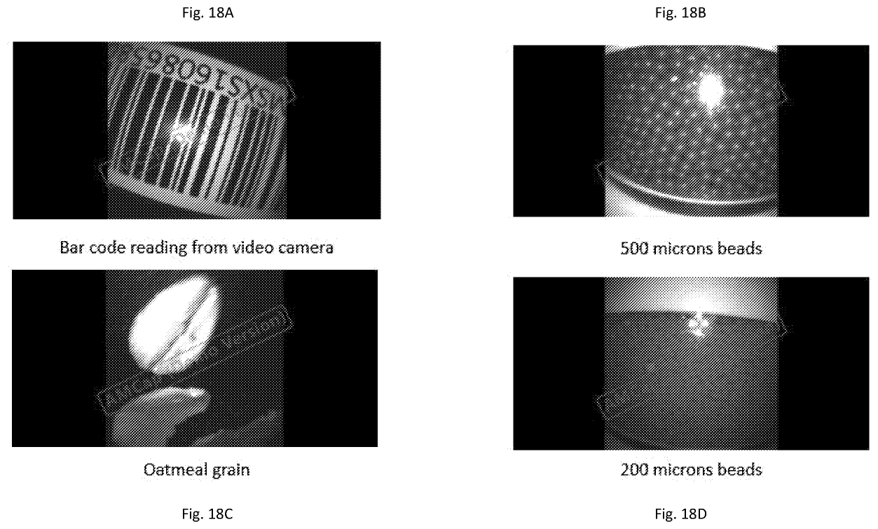

The resolution of the camera was tested to determine accuracy. The results of the test are shown in FIGS. 18A-18D. A series of products were visualized using an exemplary camera for use on the device. The photographs include a bar code (FIG. 18A), 500 micron beads (FIG. 18B), oatmeal grain (FIG. 18C), and 200 micron beads (FIG. 18D).

### EXAMPLES

### Example 1: Expandable Device Design

The expandable device is intended to capture tissue, such as i.e., esophageal cell tissue, and/or deliver drugs to a tissue by delivering abrasive strips to a target location via nasal or oral route. The device includes a catheter and expandable device, which is passed through the nose or mouth and guided to the target tissue. The abrasive strips are covered during the device insertion and removal by a unique design which hides the abrasive surface in internal folds of the expandable device. As the device is delivered through the body lumens the expandable device is maintained in a deflated form. It is then expanded 1×, 2×, 3× or 4× or more of its original size when the target tissue is reached. Because of this higher expansion requirement medical grade silicone Dow C6-530 was selected for expandable device manufacturing.

Although the medical grade silicone provides highly desirable expansion properties, it is very slippery and does not stick to many substrates. Bonding silicone is extremely challenging. Additionally the elongation property of the silicone and abrasive strips is very different. Silicone is very flexible and has very high elongation however the flexible polyolefin abrasive strip has very low elongation. Due to a significant difference in the elongation properties between the two materials it was challenging to bond them to gather and ensure device performance and size specification.

Applicant was able to solve this problem based on several surprising advances. If the abrasive strips were bonded to a silicone expandable device when the expandable device was deflated then it restricted the expandable device inflation. If the abrasive strips were bonded to the expandable device when the expandable device was inflated then it restricted expandable device deflation and some of the strips remained exposed to the outer surface.

In order to avoid these problems, abrasive strips were broken into specific length and size prior to bonding with the silicone expandable device, and the expandable device was inflated to 15 to 17 mm during the strip bonding process. To ensure symmetrical inflation to three times from its original nominal diameter with the abrasive strips along with maintaining maximum tissue contact area for abrasive strip and ensuring abrasive strips will be covered inside the balloon fold. Expandable devices shown in FIGS. 13 and 15 provide successful examples of a pattern of the strips.

### Example 2: Comparison of Different Expandable Device/Strip Configurations and Materials

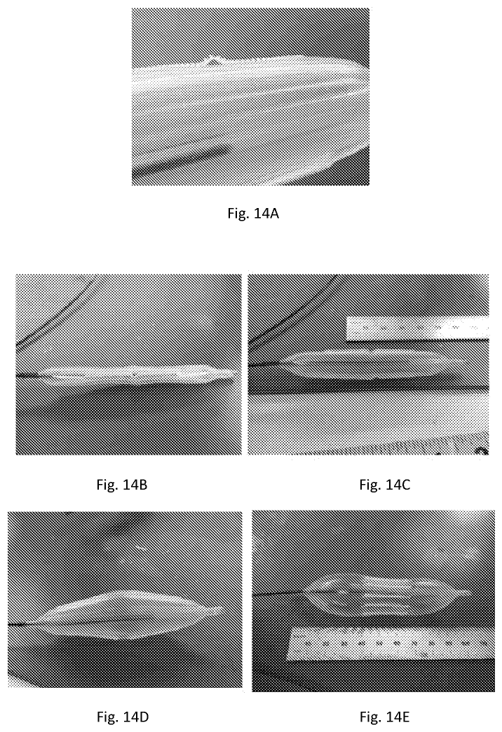

Several less desirable configurations were developed in order to design the optimal device for probing the lumens of the body and extracting tissue or delivering drugs to a subject. FIGS. 14A-14E show photographs of failed attempts to create an expandable device having a substantially cylindrical shape with strips that would have maximal exposure to the tissue in the body.

In order to eliminate any risk associated with potential biocompatibility using medical grade silicon, Dow C6-530 with Med 3-4800-7 blue color pigment was selected as a material for the expandable device.

In view of the difficultly associated with bonding anything to medical grade silicone, we first focused on bond integrity between the expandable device and the abrasive strips. Four studies were run to test the use of Loctite SI 4902, a flexible fast curing adhesive to bond the strips to the blue expandable device.

A first example involved an expandable device made using MM P/N 017561 Rev C and 84 abrasive strips MM P/N 017592 Rev A and bonded using Loctite SI 4902. The bond joint initially held up but bond integrity started degrading over time at room temperature (22 degree Celsius). The strips fell off and uncured adhesive was observed underneath them.

After trying a few other cynoacrylic adhesive options, Loctite SI 5055 UV cure adhesive proved to be successful. In order to use a UV cure adhesive either a clear expandable device and/or clear abrasive strips is important to ensure UV light penetration. A semi-clear expandable device was obtained and tested with Loctite SI 5055. The assembled expandable device was put through the following tests:
a) Multiple cycles of inflation/deflation.
b) Skin Rub test.
c) Tracking and scraping movement in a simulated esophageal cavity set up (15 mm and 20 mm tube submerged in water)
d) deflated expandable device passed through simulated mouth to esophagus curve to observed its traction

This expandable device assembly performed very well even under the worst-case scenario. All bonds stayed intact.

After rigorous testing, the Loctite SI 5055 UV cure adhesive was selected as an important adhesive.

The strip length, location, and number of strips was investigated. Initially device assembly time with a total of 84 strips was very high (5-6 hours). The purpose of the study was to achieve process optimization by:
a) Reducing labor hours to bring down cost/device
b) Using less material if possible
c) Getting optimal results as per the User needs (provide sufficient abrasion while the balloon is open, and no abrasion while the balloon is closed/deflated)

### Test Case 1:

Design: Two 52 mm long abrasive strips per fold. The construct is shown in FIG. 14A

Result: The expandable device wrinkled upon closing the balloon exposing the abrasive strips. This configuration was not desirable as the abrasive surfaces should be hidden until the balloon is inflated. Strip delamination was also observed as it was a challenge to get enough adhesive under the strips.

### Test Case 2:

Design: Two 42 mm long abrasive strips per fold in the center:

Result: The expandable device wrinkled upon closing the balloon exposing the abrasive strips. This configuration was not desirable as the abrasive surfaces should be hidden until the balloon is inflated. The constructs are shown in FIGS. 14B and 14C.

### Test Case 3:

Design: Four 25 mm long abrasive strips per fold on each side:

Result: The expandable device assumed a diamond shape upon 20 mm inflation. This configuration was not desirable as in this shape abrasive strips do not get maximum contact surface area with the tissue cells. The construct is shown in FIG. 14D.

### Test Case 4:

Design: Two 25 mm long abrasive strips per fold in the center:

Result: The expandable device assumed a dumbbell shape upon 20 mm inflation. This configuration is not acceptable as in this shape abrasive strips would not come in contact with the tissue cells. The construct is shown in FIG. 14E.

Unlike these failed constructs, the expandable device shown in FIG. 15 has a desirable conformation and is a successful device according to the invention. It has a cylindrical or sausage shaped body with tapered ends.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. Each of the foregoing patents, patent applications and references is hereby incorporated by reference.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A device, comprising
a catheter body having an outer surface sized to fit within a body lumen and extending distally from a proximal end,
an expandable device positioned over at least a portion of the outer surface, wherein when the expandable device is inflated it has a substantially cylindrical shape with tapered ends, and wherein when the expandable device is deflated the outer surface has internal folds and external folds, wherein a textured surface is present on one or more internal folds of the outer surface and is not present on one or more external folds, and wherein the textured surface is comprised of 1-50 strips of a tissue collection material on each internal fold.

2. The device of claim 1, wherein the textured surface is comprised of 2-50 strips of a textured material on each internal fold.

3. The device of claim 1, wherein the tissue collection surface is comprised of 3-9 strips of a textured material on each internal fold.

4. The device of claim 3, wherein each internal fold includes 2 side walls and a bottom wall and wherein 3 strips of textured material are present on at least one the two side walls and/or the bottom wall of each internal fold.

5. The device of claim 4, wherein each of the 3 strips are arranged linearly with a space between strips.

6. The device of claim 4, wherein two of the 3 strips are 8-15 mm in length and the third strip is 15-29 mm in length.

7. The device of claim 4, wherein two of the 3 strips are 10 mm in length and the third strip is 25 mm in length.

8. The device of claim 7, wherein the strip of 25 mm in length is positioned between the two 10 mm strips.

9. The device of claim 2, wherein the strips are made of a clear material.

10. The device of claim 2, wherein the strips are polyolefin low profile hook strips.

11-67. (canceled)

68. A disposable multiple lumen device, comprising
a flexible catheter body having an outer surface sized to fit within a body lumen and extending distally from a proximal end to a distal end, wherein the catheter body has at least two lumens, each having a distal opening at or near the distal end of the catheter, wherein the first lumen is a hollow compartment for transferring components from the proximal to distal ends of the catheter, the second lumen being a flushing channel,
a visualization device, optionally a camera, positioned on the catheter distal to the expandable device, and
a light source in proximity to the visualization device.

69. The disposable multiple lumen device of claim 68, further comprising a handle fixed to the proximal end of the flexible catheter body.

70. The disposable multiple lumen device of claim 69, wherein the handle includes a housing that encloses a steering mechanism comprising a rotating toothed gear.

71. The disposable multiple lumen device of claim 70, wherein the handle housing contains a control element, wherein a portion of the control element is exposed to a proximal end of the handle such that the control element can be manipulated single-handedly.

72. The disposable multiple lumen device of claim 70, wherein the flexible catheter body is detachably and directly connected at its proximal end to a distal end of the handle and includes at least one guide wire disposed therein, wherein the at least one guide wire extends from the proximal end of the flexible catheter body and is constructed to fit into a distal end portion of the steering mechanism housed in the handle such that the at least one guide wire can be manipulated by the steering mechanism to move the flexible catheter body in one of at least four different directions.

73. The disposable multiple lumen device of claim 69, wherein the handle does not include any gears.

74. The disposable multiple lumen device of claim 69, wherein the handle is disposable.

75-83. (canceled)

84. The disposable multiple lumen device of claim 70, further comprising an irrigation/suction valve positioned on the handle housing and operably connected to the second lumen.

85. A method of treating an individual, comprising:
(a) advancing a disposable multiple lumen device of claim 68 into a tissue selected from an esophagus, pharynx, larynx, trachea, urethra, bladder, colon, cervix, or duodenum of the individual;
(b) visualizing a delivery site in the tissue using the visualization device and light source;
(c) treating the delivery site by delivering through the first lumen of the catheter body an agent or a surgical instrument to the delivery site in order to treat the individual; and
(d) removing the disposable multiple lumen device from the individual.

86. The method of claim 85, wherein the agent is a therapeutic agent, a diagnostic agent or an imaging agent.

87. The method of claim 85, wherein the step of treating the delivery site involves the delivery of both an agent and a surgical instrument.
